(19) United States
(12) Patent Application Publication
(10) Pub. No.: US 2024/0347180 A1
(43) Pub. Date: Oct. 17, 2024

(54) METHOD AND APPARATUS FOR RESPONDING TO DECONTAMINATION SITUATIONS

(71) Applicants: Christopher Maze, Nampa, ID (US); Kara Maze, Nampa, ID (US)

(72) Inventors: Christopher Maze, Nampa, ID (US); Kara Maze, Nampa, ID (US)

(21) Appl. No.: 18/135,723

(22) Filed: Apr. 17, 2023

(51) Int. Cl. G16H 40/20; G16H 50/80

(52) U.S. Cl. CPC G16H 40/20; G16H 50/80

(57) ABSTRACT

A Rapid Communication System stored on a non-transitory storage medium may be communicatively-coupled to a processor; the non-transitory storage medium may include a first user data receiver to receive, from a first team member's device, first team member data, the first team member data identifying attributes of a first team member user.

### BACKGROUND

Hospitals often have a need for individuals who are trained and available to perform decontamination of one or more individuals which have been contacted by at least one hazardous materials, such as a hazardous chemical.

Under the laws of various jurisdictions, individuals who participate in decontamination exercises may be required to wear personal protective equipment while performing decontamination. Various OSHA regulations often contribute to the difficulty of responding to contamination events in a safe, quick and compliant manner. A need exists for finding individuals with legal, up-to-date credentials who can rapidly travel to a contamination event location, such as a hospital, and assist with decontamination of individuals or the environment.

A need also exists for a system that is useful for coordinating the efforts of response individuals as well as tracking compliance efforts and reminding response individuals to comply with legal requirements governing decontamination by humans.

### SUMMARY

According to one example, a rapid communication system for communicating with decontamination team members is disclosed; the system may also send messages to hospital personnel. The rapid communication system may record the response time of team members, such as by measuring the moment when team members are notified of a contamination event, measuring the moment when team members send a message via the system that the one or more team members are available to respond to the contamination event, measuring the geo-coordinates of team members so as to track the time interval between the first message about a contamination event that has been sent to a specific team member and when the team member actually brings a device having a rapid responder tracking system and broadcasting the device's current geo-location.

The rapid communication system may also record the team member or team members response time as recorded by a team member making a selection or inputting a response time. The rapid communication system may determine whether a team member is able to respond to a contamination event. The rapid communication system may receive notes inputted by team members, store the date and time when the notes were inputted via a rapid responder tracking system, associate the notes with a team member unique identifier, and store the notes to a database so that the notes may be retrieved by the system and formatted for a report. The system may create a unique identifier; the unique identifier may be associated with a one or more pieces of personal protective equipment; the unique identifier may be associated with one or more pieces of personal protective equipment and one or more team members. In a preferred embodiment, the unique identifier may be associated with a set of persona protective equipment and a single team member. The system may format the unique identifier as a barcode, QR code, or other scannable image, thereby creating a formatted unique identifier; the system may then send the formatted unique identifier to a printer or other device.

When a team member selects or responds to a request for a response to a contamination event, the rapid communication system may access the geo-coordinates for the team member as well as the geo-coordinates for the location of the contamination event; the rapid communication system may use an algorithm to measure the distance between the team member's present location and the location of the contamination event and calculate the estimated travel time, thereby generating an estimated time of arrival for the team member. The rapid communication system may request and receive data from providers, such as Google Maps, providing information about real-time traffic conditions;
the rapid communication system may incorporate data about real-time traffic conditions when determining the estimated time of arrival; the rapid communication system may send messages to a plurality of team members, receive responses from a plurality of team members, calculate the estimated time of arrival for a plurality of team members, send a message to the team members associated with the smallest estimated time of arrival wherein the message requests the rapid response of said team member.

In the preferred methods of use, a user may retrieve a formatted unique identifier from the system; the user may then cause the formatted unique identifier to be printed onto a sticker; the user may then affix the formatted unique identifier onto a piece of personal protective equipment.

In some embodiments, a method of use includes a computer system receiving data; wherein the data contains information about a piece of personal protective equipment and a team member; another step of the method may be for the computer system to record in a database the date and time when a device was first used to optically-scan a formatted unique identifier; the method may also include a step of recording in the database the date and time was the device was used a second time to optically-scan the formatted unique identifier. The system may calculate the length of time between the first optical scan and the second optical scan, thereby calculating “Time In” and “Time Out” for wearing the personal protective equipment. When a formatted unique identifier is scanned by a computer system, then the computer system may begin using one or more sensors to measure vital signs on a team member or may display instructions on a screen or by playing audio instructions for the team member or an associate of the team member to measure the team member's vital signs, preferably before the personal protective equipment is donned and also after the personal protective equipment has been removed from the team member. The rapid responder tracking system may instruct a computing device to play an alarm or other reminder when the rapid responder tracking system has not detected data representing a formatted unique identifier sent via a scanning camera of a computing device associated with the rapid responder tracking system.

The systems may have one or more modules. For purposes of this disclosure, a module comprises a combination of one or more processors in communication with at least one non-transitory storage device storing instructions.

### BRIEF DESCRIPTION OF THE SEVERAL VIEWS OF THE DRAWINGS

FIG. 1 is a Rapid Communication System operating a rapid communication network, according to an example of the principles described herein. Rapid Communication System 110 is depicted communicating with a computing device 105.

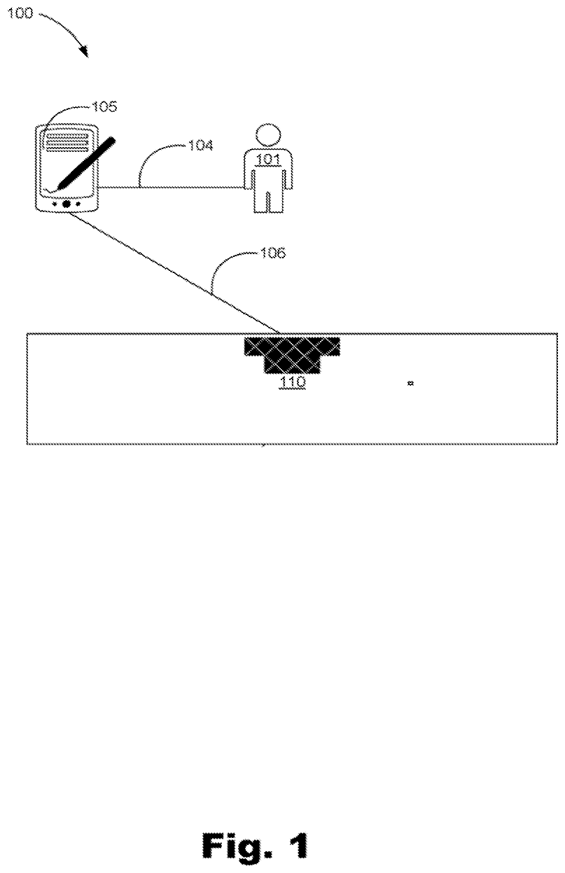

The Rapid Responder Tracking System 600 may be implemented on an electronic device. Examples of electronic devices include servers, desktop computers, laptop computers, personal digital assistants (PDAs), mobile devices, smartphones, gaming systems, and tablets, among other electronic devices.

The Rapid Responder Tracking System 600 may be utilized in any data processing scenario, including stand-alone hardware, mobile applications, a computing network, or combinations thereof. Further, the rapid responder tracking system 600 may be used in a computing network, a public cloud network, a private cloud network, a hybrid cloud network, other forms of networks, or combinations thereof. In one example, the methods provided by the rapid responder tracking system 600 are provided as a service over a network by, for example, a third party. In this example, the service may comprise, for example, the following: a Software as a Service (SaaS) hosting a number of applications; a Platform as a Service (PaaS) hosting a computing platform comprising, for example, operating systems, hardware, and storage, among others; an Infrastructure as a Service (IaaS) hosting equipment such as, for example, servers, storage components, network, and components, among others; application program interface (API) as a service (APIaaS), other forms of network services, or combinations thereof. The present systems may be implemented on one platform or multiple hardware platforms, in which the modules in the system can be executed on one platform or across multiple platforms. Such modules can run on various forms of cloud technologies and hybrid cloud technologies or offered as a Saas (Software as a Service) that can be implemented on or off the cloud. In another example, the methods provided by the rapid responder tracking system 600 are executed by a local administrator.

The rapid responder tracking system 600 includes a processing resource 602 which may include the hardware architecture to retrieve executable code from the Rapid Communication System 610 and execute the executable code. The executable code may, when executed by the processor 602, cause the processor 602 to implement at least the functionality of operating a Rapid Communication System 610, according to the methods of the present specification described herein. In the course of executing code, the processor 602 may receive input from and provide output to a number of the remaining hardware units.

Rapid Communication System 610 may store data such as executable program code that is executed by the processor 602 or other processing device. As will be discussed, the Rapid Communication System 610 may specifically store computer code representing a number of applications that the processor 602 executes to implement at least the functionality described herein.

The Rapid Communication System 610 further comprises a number of modules used in the implementation of communicating information between team members and the Rapid Communication System 610. The various modules within the Rapid Communication System 610 may comprise executable program code that may be executed separately. In this example, the various modules may be stored as separate computer program products. In another example, the various modules within the Rapid Communication System 610 may be combined within a number of computer program products; each computer program product comprising a number of the modules.

The Rapid Communication System 610 may include a first user data receiver (620-1) to receive, from a first team member's device, first team member data, the first team member data identifying attributes of a first team member user.

A non-transitory storage medium 709 includes a number of modules 720. Each module 720 may consist of a combination of hardware and software. Each module may include a processor capable of executing computer program code. The processor associated with each module may cause the module to execute computer program code to perform a particular task. The computer program code may include instructions stored as part of software or hardware.

### CROSS-REFERENCE TO RELATED APPLICATIONS

This application claims the benefit and incorporates by reference a provisional patent application, which is cited in the ADS.

What is claimed:

1. A non-transitory storage medium communicatively-coupled to a processor, the non-transitory storage medium comprising a first user data receiver to receive, from a first team member's device, first team member data, the first team member data identifying attributes of a first team member user.
